(19) United States
(12) Patent Application Publication
(10) Pub. No.: US 2004/0157796 A1
(43) Pub. Date: Aug. 12, 2004

(54) Concentrated liquid valdecoxib composition

(76) Inventor: Yatin Gokarn, Thousand Oaks, CA (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/721,645

(22) Filed: Nov. 25, 2003

Related U.S. Application Data
(60) Provisional application No. 60/429,686, filed on Nov. 27, 2002.

(51) Int. Cl. A61K 31/724; A61K 31/415

(52) U.S. Cl. 514/58; 514/406

(57) ABSTRACT

The present invention provides a pharmaceutical composition comprising a liquid carrier, valdecoxib and cyclodextrin derivative in an amount of at least about 5%, w/v, of the composition. The valdecoxib is in solubilized form and, at room temperature, the ratio of valdecoxib concentration to cyclodextrin concentration is greater than is achievable in a substantially similar solution but which comprises less than 5%, w/v, of said cyclodextrin derivative.

This application claims priority of U.S. Provisional Application Serial No. 60/429,686 filed on 27 Nov. 2002.

### FIELD OF THE INVENTION

The present invention relates to liquid formulations, for example parenterally deliverable formulations, of the selective cyclooxygenase-2 inhibitory drug, valdecoxib.

### BACKGROUND OF THE INVENTION

Liquid drug formulations, for example parenteral or imbibable formulations, have become a very important component in the arsenal of available drug delivery options, particularly for drugs having analgesic effect. In some situations, parenteral routes of administration, including subcutaneous, intramuscular and intravenous injection, are particularly advantageous. For example, parenteral administration of a drug typically results in attainment of a therapeutically effective blood serum concentration of the drug in a shorter time than is achievable by oral administration. This is especially true of intravenous injection, whereby the drug is placed directly in the bloodstream. Parenteral administration can also result in more predictable blood serum concentrations of a drug, because losses in the gastrointestinal tract due to metabolism, binding to food and other causes are eliminated. For similar reasons, parenteral administration often permits dose reduction. Parenteral administration is generally the preferred method of drug delivery in emergency situations, and is also useful in treating subjects who are uncooperative, unconscious, or otherwise unable or unwilling to accept oral medication.

If a parenteral drug formulation is to be prepared, it is preferable from patient convenience and safety standpoints that such a formulation be a ready-to-use formulation, i.e. one that does not require dilution or mixing immediately prior to use (as opposed to a reconstitutable formulation). Such formulations also avoid the need for time consuming aseptic manipulation prior to administration. Ready-to-use parenteral formulations can also be advantageous from a manufacturing standpoint by avoiding expensive lyophilization and/or other similar manufacturing steps. It is also preferable, from manufacturing, patient compliance and regulatory compliance standpoints, that such a formulation comprise minimal amounts of non-therapeutic excipients (e.g. solubilizers, preservatives, etc).

U.S. Pat. No. 5,932,598 to Talley et al. discloses a class of water-soluble prodrugs of selective COX-2 inhibitory drugs, including the compound N-[[4-(5-methyl-3-phenylisoxazol-4-yl)phenyl]sulfonyl]propanamide, also referred to herein as parecoxib (I), and salts thereof, for example the sodium salt, referred to herein as parecoxib sodium. Parecoxib sodium is currently under development by Pharmacia Corp. for, inter alia, treatment of acute pain, for example post-surgical pain.

Parecoxib, which converts to the substantially water-insoluble selective COX-2 inhibitory drug valdecoxib following administration to a subject, itself shows weak in vitro inhibitory activity against both COX-1 and COX-2, while valdecoxib (II) has strong inhibitory activity against COX-2 but is a weak inhibitor of COX-1.

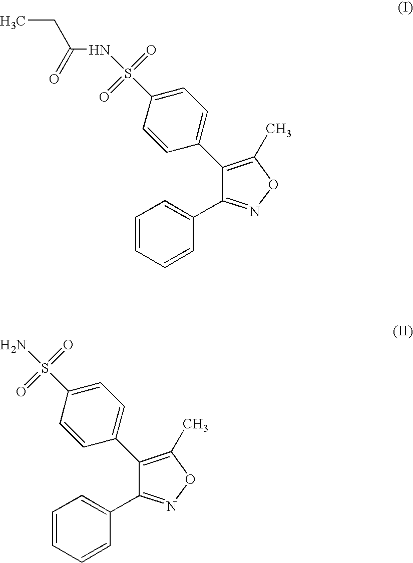

As indicated above, valdecoxib has extremely low solubility in water and, for this reason, it has been proposed to administer parenterally the much more soluble prodrug, parecoxib, that cleaves to form valdecoxib. See for example Dionne (1999), “COX-2 inhibitors—IBC Conference, 12-13 Apr. 1999, Coronado, Calif., U.S.A.”, IDrugs, 2(7), 664-666. However, it would also be beneficial to have a parenterally deliverable dosage form of valdecoxib that provides a satisfactory valdecoxib concentration and yet does not contain undesirably high quantities of non-therapeutic excipients.

U.S. Pat. No. 5,633,272 discloses that its subject isoxazolyl benzenesulfonamides, of which valdecoxib is an example, can be administered parenterally as a solution in a range of solvents including polyethylene glycol and propylene glycol. However, in order to provide a suitable valdecoxib concentration, high doses of such non-aqueous solvents would need to be used thereby rendering the formulation undesirable in many situations, for example for parenteral or ophthalmic use—situations where high concentrations of such solvents are undesirable.

Numerous references including Okimoto et al., Pharm. Res. 13:256-264; Loftsson and Brewster, J. Pharm. Sci. 85:1017-1025; U.S. Pat. No. 5,134,127 to Stella et al.; and U.S. Pat. No. 6,407,079 to Müller et al., teach compositions of cyclodextrins with drugs. However, the interaction or solubilizing capacity of a given cyclodextrin/drug combination is generally unpredictable. Furthermore, as many cyclodextrins are very expensive excipients and/or are unsuitable for parenteral use due to toxicity issues, their use has been limited.

U.S. Pat. No. 6,133,248 to Stella discloses use of cyclodextrins to solubilize small quantities of poorly water soluble degradant in the presence of high concentrations of prodrug. Moreover, U.S. patent application Publication Ser. No. 2002/0128267 to Bandyopadhyay et al., discloses that ophthalmic formulations of cyclooxygenase-2 inhibitory drugs can optionally comprise cyclodextrins.

If a liquid valdecoxib formulation having a suitable therapeutic valdecoxib concentration but which does not contain undesirably elevated concentrations of non-aqueous solubilizers could be prepared, a significant advance in the art would be realized.

### SUMMARY OF THE INVENTION

The present invention provides a pharmaceutical composition comprising a liquid carrier, valdecoxib, and a cyclodextrin wherein the cyclodextrin is in an amount of not less than 5% by weight of the composition volume (“w/v”) and preferably not less than about 7.5%, w/v. At least a substantial portion of the valdecoxib is in solubilized form in the liquid carrier and, at a given temperature, the weight ratio of the valdecoxib in solubilized form to the cyclodextrin is in a portion greater than is achievable at the same temperature in a substantially similar composition but which comprises less than 5%, w/v, of the same cyclodextrin. Unless context instructs otherwise, the term “w/v” means weight of a component per volume of the composition. For convenience to the reader, sometimes the term “w/v” is followed by the term “of the composition”.

Also provided by the present invention are methods for treating or preventing a cyclooxygenase-2 mediated disorder or condition in s subject in need of such treatment.

The instant invention provides a liquid valdecoxib composition capable of achieving unexpectedly high valdecoxib concentrations while utilizing surprisingly low amounts of non-aqueous solubilizing agent and, therefore, represents a significant advance in the art.

### BRIEF DESCRIPTION OF THE DRAWINGS

FIG. 1 shows equilibrium solubility of valdecoxib as a function of sulfobutyl ether_{6.4}-β-cyclodextrin (SBE_{6.4} CD) concentration (% w/v) at 25° C.

FIG. 2 shows equilibrium solubility of valdecoxib as a function of hydroxypropyl-β-cyclodextrin (HP-β-CD) concentration (% w/v) at 25° C.

FIG. 3 shows a plot of valdecoxib versus cyclodextrin concentration in each of two dilute samples comprising hydroxypropyl-β-cyclodextrin or sulfobutyl ether_{6.4}-β-cyclodextrin plus valdecoxib.

### DETAILED DESCRIPTION OF THE INVENTION

### Valdecoxib

A composition of the invention comprises valdecoxib at a concentration of at least about 1 mg/ml and preferably at least about 2 mg/ml, for example about 1 mg/ml to about 20 mg/ml, preferably about 2 mg/ml to about 15 mg/ml, and more preferably about 4 mg/ml to about 10 mg/ml. Valdecoxib suitable for use in a composition of the invention can be prepared by any suitable process, illustratively by processes described in U.S. Pat. No. 5,633,272 to Talley et al. At least a substantial portion of the valdecoxib present in a composition of the invention is in solubilized form. Preferably at least substantially all of the valdecoxib present in a composition of the invention is in solubilized form.

### Cyclodextrin

A composition of the invention comprises at least one cyclodextrin, also referred to herein as a cyclodextrin derivative. Cyclodextrins suitable for use in a composition of the invention can be α-cyclodextrins or β-cyclodextrins (also referred to herein as β-CD). Preferably the cyclodextrins are β-cyclodextrins.

In one embodiment, the cyclodextrin is a partially etherified β-cyclodextrin, substantially as is described in U.S. Pat. No. 6,407,079 to Muller et al., of formula (III):

(β-CD)—(OR)₂₁ (III)

wherein R groups are independently selected from hydrogen, hydroxyalkyl or alkyl and wherein at least one R group is hydroxyalkyl. Preferably, the at least one hydroxyalkyl group is hydroxyethyl, hydroxypropyl or dihydroxypropyl. Preferred alkyl groups are methyl and/or ethyl groups.

β-cyclodextrin is a compound with ring structure consisting of 7 anhydro glucose units; it is also referred to as cycloheptaamylose. Each of the 7 glucose rings contains in 2-, 3-, and 6-position three hydroxy groups which may be etherified. Therefore, a total of 21 hydroxy groups per cyclodextrin molecule are available for etherification. In the partially etherified β-cyclodextrin derivatives suitable for the present invention only a portion of these available hydroxy groups are etherfied with hydroxyalkyl groups. Optionally a portion of these available hydroxy groups are etherfied with alkyl groups. In the hydroxyalkyl ethers of β-cyclodextrin used in accordance with the invention the average degree of substitution (DS) with hydroxyalkyl groups per cyclodextrin molecule is preferably about 0.5 to about 20, more preferably about 2 to about 18 and still more preferably about 3 to about 16.

Partially etherified β-cyclodextrin which comprises, in addition to hydroxyalkyl groups, alkyl groups, preferably have a degree of substitution per cyclodextrin molecule of about 0.35 to about 16 and preferably about 1.4 to about 15.

Especially preferred cyclodextrins are hydroxyethyl, hydroxypropyl and dihydroxypropyl ether cyclodextrins, their corresponding mixed ethers, and further mixed ethers with methyl or ethyl groups, such as methyl-hydroxyethyl, methyl-hydroxypropyl, ethyl-hydroxyethyl, and ethyl-hydroxypropyl ether of β-cyclodextrin.

Preparation of hydroxyalkyl ethers of β-cyclodextrin can be carried out using any suitable method, for example methods described in U.S. Pat. No. 3,459,731 to Gramera et al.

In another embodiment, the β-cyclodextrin is a partially alkylated β-cyclodextrin, for example a partially methylated or partially dimethylated β-cyclodextrin. Partially alkylated β-cyclodextrins preferably have an average degree of substitution (DS) with alkyl groups per cyclodextrin molecule of about 0.5 to about 20, more preferably about 2 to about 18 and still more preferably about 3 to about 16, for example about 14.

In another embodiment, the cyclodextrin is selected from those described in U.S. Pat. No. 5,134,127 and has a structure represented by formula (IV):

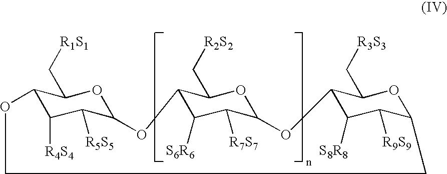

Wherein:

N is 4, 5, or 6;

R₁, R₂ R₃, R₄, R₅, R₆, R₇, R₈ and R₉ are each, independently, O⁻ or a O—C₂₋₆-alkylene)-SO₃⁻ group, wherein at least one of R₁ and R₂ is independently a O—(C₂₋₆-alkylene)-SO₃⁻ group, preferably a O—(CH₂)ₘSO₃⁻ group, wherein m is 2 to 6, preferably 2 to 4, (e.g. OCH₂CH₂CH₂SO₃⁻ or OCH₂CH₂CH₂CH₂SO₃⁻); and S₁, S₂, S₃, S₄, S₅, S₆, S₇, S₈, and S₉ are each, independently, a pharmaceutically acceptable cation which includes, for example, H⁺, alkali metals (e.g. Li⁺, Na⁺, K⁺), alkaline earth metals (e.g., Ca⁺², Mg⁺²), ammonium ions and amine cations such as the C₁₋₆ alkylamines, piperidine, pyrazine, C₁₋₆ alkanolamine and C₄₋₈ cycloalkanolamine.

In a preferred embodiment, R₁ is a O—(C₂₆-alkylene)-SO₃⁻ group, more preferably a O—(CH₂)mSO₃⁻ group (e.g. OCH₂CH₂CH₂SO₃⁻ or OCH₂CH₂CH₂CH₂SO₃) wherein m is 2-6, preferably 2-4;

R₂ to R₉ are O⁻;

S₁ to S₉ are each, independently, a pharmaceutically acceptable cation.

In another preferred embodiment, R₁, R₂ and R₃ are each, independently, a O—(C₂₋₆-alkylene)-SO₃⁻ group, preferably a O—(CH₂)ₘSO₃⁻ group, (e.g. OCH₂CH₂CH₂SO₃⁻ or OCH₂CH₂CH₂CH₂SO₃⁻) wherein m is 2-6, preferably 2-4;

R₄ to R₉ are O⁻; and

S₁ to S₉ are each, independently, a pharmaceutically acceptable cation.

In yet another preferred embodiment, R₁ to R₃ are each, independently, a O—(C₂₋₆-alkylene)-SO₃⁻ group;

at least one of R₄, R₆ and R₈ is a O—(C₂₋₆-alkylene)-SO₃⁻ group, preferably a O—(CH₂)ₘSO₃— group wherein m is 2-6, preferably 2-4 (e.g., OCH₂CH₂CH₂SO₃⁻ or OCH₂CH₂CH₂CH₂SO₃⁻);

R₅, R₇ and R₉ are O⁻; and

S₁ to S₉ are each, independently, a pharmaceutically acceptable cation.

In another preferred embodiment:

R₁, R₂, R₃, R₄, R₆ and R₈ are each, independently, a O—(C₂₋₆-alkylene)-SO₃⁻ group, preferably a O—(CH₂)mSO₃— group wherein m is 2-6, preferably 2-4 (e.g. OCH₂CH₂CH₂CH₂SO₃⁻ or OCH₂CH₂CH₂SO₃⁻);

R₅, R₇ and R₉ are O⁻; and

S₁ to S₉ are each, independently, a pharmaceutically acceptable cation.

Preferred among these cyclodextrin derivatives are those wherein the C₂₋₆ alkylene is a C₃ or C₄ alkylene. A particularly preferred cyclodextrin is sulfoalkylether β-cyclodextrin, for example sulfobutylether-β-cyclodextrin having an average substitution of about 4 to about 8 and preferably about 5 to about 7, for example about 6.4 sulfobutyl ether linkages (i.e. sulfobutyl ether_{6.4}-β-cyclodextrin).

### Unexpected Valdecoxib-cyclodextrin Ratio

One or more cyclodextrins are present in a composition of the invention in an amount of at least about 5%, preferably at least about 7.5%, more preferably at least about 10%, still more preferably at least about 12.5%, yet more preferably at least about 15%, and even more preferably at least about 20%. Illustratively, a cyclodextrin is present in a total amount of about 5% to about 95%, preferably about 5% to about 80% or about 7.5% to about 75% or about 10% to about 60%, more preferably about 15% to about 50%, or about 20% to about 50% (w/v).

Surprisingly, we have now discovered that, in general, as cyclodextrin derivative concentration in a composition of the invention increases, the achievable ratio of solubilized valdecoxib to cyclodextrin derivative also increases. Without being bound by theory, it is believed that such additional unexpected valdecoxib solubility is due, at least in part, to non-ideal behavior of the solvent system leading to a subsequent co-solvent-like effect, rather than to any higher order binding (e.g. 1:2 valdecoxib:cyclodextrin binding). This unexpected finding indicates the possibility for preparing a composition at a given valdecoxib concentration using less cyclodextrin derivative than was heretofore expected, which is advantageous at least from cost and regulatory compliance perspectives. This surprising finding also indicates the possibility for preparing a composition at a given cyclodextrin derivative concentration which achieves a higher valdecoxib concentration than heretofore believed possible. Since valdecoxib is a drug of very poor water solubility, this discovery provides a way to administer valdecoxib in parenterally feasible volumes of liquid.

In a preferred embodiment, a cyclodextrin is present in an amount of at least about 7.5% w/v and the weight ratio of solubilized valdecoxib to cyclodextrin in the composition is in a proportion of at least about 2.5%, preferably at least about 5%, and more preferably at least about 10% greater than is achievable at the same temperature in a substantially similar composition but which comprises less than 5% w/v cyclodextrin, for example 2.5% w/v cyclodextrin. Illustratively, if the weight ratio of solubilized valdecoxib (0.221 mg/ml composition) to cyclodextrin in a composition containing 2.5% (w/v) cyclodextrin is 8.84 (i.e. 0.221 mg valdecoxib/ml composition per 0.025 mg cyclodextrin/ml composition=8.84), the weight ratio of solubilized valdecoxib to cyclodextrin in a composition of this embodiment will be at least about 2.5% greater than 8.84 (i.e. at least about 9.06).

It will be understood that in performing a test to determine whether the ratio of solubilized valdecoxib to cyclodextrin in a composition is greater than is achievable in a substantially similar composition but which comprises less than 5% cyclodextrin, the test will be performed at substantially the same temperature and under substantially identical conditions for both an inventive composition and for any comparative compositions.

In another particularly preferred embodiment a cyclodextrin derivative is present in an amount of at least about 10% w/v and the ratio of solubilized valdecoxib to cyclodextrin in the composition is in a proportion at least about 5%, more preferably at least about 10%, and still more preferably at least about 15% greater than is achievable in a substantially similar composition at the same temperature but which comprises less than 5% cyclodextrin w/v, for example 2.5% cyclodextrin w/v.

While each of the elements of the present invention are described herein as containing multiple embodiments, it should be understood that, unless indicated otherwise, each of the embodiments of a given element of the present invention are capable of being used with each of the embodiments of the other elements of the present invention and each such use is intended to form distinct embodiment of the present invention. For the sake of clarity, the term “elements of the present invention” as used herein in, includes a liquid carrier, valdecoxib, and a cyclodextrin.

### Method of Treatment

Compositions of the invention are useful in treatment and prevention of a very wide range of disorders mediated by COX-2, including but not restricted to disorders characterized by inflammation, pain and/or fever. Such compositions are especially useful as anti-inflammatory agents, such as in treatment of arthritis, with the additional benefit of having significantly less harmful side effects than compositions of conventional nonsteroidal anti-inflammatory drugs (NSAIDs) that lack selectivity for COX-2 over COX-1. In particular, compositions of the invention have reduced potential for gastrointestinal toxicity and gastrointestinal irritation including upper gastrointestinal ulceration and bleeding, reduced potential for renal side effects such as reduction in renal function leading to fluid retention and exacerbation of hypertension, reduced effect on bleeding times including inhibition of platelet function, and possibly a lessened ability to induce asthma attacks in aspirin-sensitive asthmatic subjects, by comparison with compositions of conventional NSAIDs. Thus compositions of the invention are particularly useful as an alternative to conventional NSAIDs where such NSAIDs are contraindicated, for example in patients with peptic ulcers, gastritis, regional enteritis, ulcerative colitis, diverticulitis or with a recurrent history of gastrointestinal lesions; gastrointestinal bleeding, coagulation disorders including anemia such as hypoprothrombinemia, hemophilia or other bleeding problems; kidney disease; or in patients prior to surgery or patients taking anticoagulants.

Contemplated compositions are useful to treat a variety of arthritic disorders, including but not limited to rheumatoid arthritis, spondyloarthropathies, gouty arthritis, osteoarthritis, systemic lupus erythematosus and juvenile arthritis.

Such compositions are useful in treatment of asthma, bronchitis, menstrual cramps, preterm labor, tendonitis, bursitis, allergic neuritis, cytomegalovirus infectivity, apoptosis including HIV-induced apoptosis, lumbago, liver disease including hepatitis, skin-related conditions such as psoriasis, eczema, acne, burns, dermatitis and ultraviolet radiation damage including sunburn, and post-operative inflammation including that following ophthalmic surgery such as cataract surgery or refractive surgery.

Such compositions are useful to treat gastrointestinal conditions such as inflammatory bowel disease, Crohn's disease, gastritis, irritable bowel syndrome and ulcerative colitis.

Such compositions are useful in treating inflammation in such diseases as migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like.

Such compositions are useful in treatment of ophthalmic diseases, such as retinitis, conjunctivitis, retinopathies, uveitis, ocular photophobia, and of acute injury to the eye tissue.

Such compositions are useful in treatment of pulmonary inflammation, such as that associated with viral infections and cystic fibrosis, and in bone resorption such as that associated with osteoporosis.

Such compositions are useful for treatment of certain central nervous system disorders, such as cortical dementias including Alzheimer's disease, neurodegeneration, and central nervous system damage resulting from stroke, ischemia and trauma. The term “treatment” in the present context includes partial or total inhibition of dementias, including Alzheimer's disease, vascular dementia, multi-infarct dementia, pre-senile dementia, alcoholic dementia and senile dementia.

Such compositions are useful in treatment of allergic rhinitis, respiratory distress syndrome, endotoxin shock syndrome and liver disease.

Such compositions are particularly useful in treatment of pain, including but not limited to postoperative pain, dental pain, muscular pain, and pain resulting from cancer. For example, such compositions are useful for relief of pain, fever and inflammation in a variety of conditions including rheumatic fever, influenza and other viral infections including common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, burns, and trauma following surgical and dental procedures.

Such compositions are useful for treating and preventing inflammation-related cardiovascular disorders, including vascular diseases, coronary artery disease, aneurysm, vascular rejection, arteriosclerosis, atherosclerosis including cardiac transplant atherosclerosis, myocardial infarction, embolism, stroke, thrombosis including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

Such compositions are useful in treatment of angiogenesis-related disorders in a subject, for example to inhibit tumor angiogenesis. Such compositions are useful in treatment of neoplasia, including metastasis; ophthalmological conditions such as corneal graft rejection, ocular neovascularization, retinal neovascularization including neovascularization following injury or infection, diabetic retinopathy, macular degeneration, retrolental fibroplasia and neovascular glaucoma; ulcerative diseases such as gastric ulcer; pathological, but non-malignant, conditions such as hemangiomas, including infantile hemaginomas, angiofibroma of the nasopharynx and avascular necrosis of bone; and disorders of the female reproductive system such as endometriosis.

Such compositions are useful in prevention and treatment of benign and malignant tumors and neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophogeal cancer, small bowel cancer, stomach cancer, colon cancer, liver cancer, bladder cancer, pancreas cancer, ovary cancer, cervical cancer, lung cancer, breast cancer, skin cancer such as squamous cell and basal cell cancers, prostate cancer, renal cell carcinoma, and other known cancers that effect epithelial cells throughout the body. Neoplasias for which compositions of the invention are contemplated to be particularly useful are gastrointestinal cancer, Barrett's esophagus, liver cancer, bladder cancer, pancreatic cancer, ovarian cancer, prostate cancer, cervical cancer, lung cancer, breast cancer and skin cancer. Such compositions can also be used to treat fibrosis that occurs with radiation therapy. Such compositions can be used to treat subjects having adenomatous polyps, including those with familial adenomatous polyposis (FAP). Additionally, such compositions can be used to prevent polyps from forming in patients at risk of FAP.

Such compositions inhibit prostanoid-induced smooth muscle contraction by inhibiting synthesis of contractile prostanoids and hence can be of use in treatment of dysmenorrhea, premature labour, asthma and eosinophil-related disorders. They also can be of use for decreasing bone loss particularly in postmenopausal women (i.e., treatment of osteoporosis), and for treatment of glaucoma.

Preferred uses for compositions of the invention are for treatment of rheumatoid arthritis and osteoarthritis, for pain management generally (particularly post-oral surgery pain, post-general surgery pain, post-orthopedic surgery pain, and acute flares of osteoarthritis), for treatment of Alzheimer's disease, and for colon cancer chemoprevention.

Besides being useful for human treatment, compositions of the invention are useful for veterinary treatment of companion animals, exotic animals, farm animals, and the like, particularly mammals. More particularly, compositions of the invention are useful for treatment of COX-2 mediated disorders in horses, dogs and cats.

The present invention is further directed to a therapeutic method of treating a condition or disorder where treatment with a COX-2 inhibitory drug is indicated, the method comprising administration of a composition of the invention to a subject in need thereof, for example orally or parenterally. The dosage regimen to prevent, give relief from, or ameliorate the condition or disorder preferably corresponds to once-a-day or twice-a-day treatment, but can be modified in accordance with a variety of factors. These include the type, age, weight, sex, diet and medical condition of the subject and the nature and severity of the disorder. Thus, the dosage regimen actually employed can vary widely.

Initial treatment can begin with a dose regimen as indicated above. Treatment is generally continued as necessary over a period of several weeks to several months or years until the condition or disorder has been controlled or eliminated. Subjects undergoing treatment with a composition of the invention can be routinely monitored by any of the methods well known in the art to determine effectiveness of therapy. Continuous analysis of data from such monitoring permits modification of the treatment regimen during therapy so that optimally effective doses are administered at any point in time, and so that the duration of treatment can be determined. In this way, the treatment regimen and dosing schedule can be rationally modified over the course of therapy so that the lowest amount of the composition exhibiting satisfactory effectiveness is administered, and so that administration is continued only for so long as is necessary to successfully treat the condition or disorder.

The present compositions can be used in combination therapies with opioids and other analgesics, including narcotic analgesics, Mu receptor antagonists, Kappa receptor antagonists, non-narcotic (i.e. non-addictive) analgesics, monoamine uptake inhibitors, adenosine regulating agents, cannabinoid derivatives, Substance P antagonists, neurokinin-1 receptor antagonists and sodium channel blockers, among others. Preferred combination therapies comprise use of a composition of the invention with one or more compounds selected from aceclofenac, acemetacin, e-acetamidocaproic acid, acetaminophen, acetaminosalol, acetanilide, acetylsalicylic acid (aspirin), S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, α-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lornoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen and zomepirac (see The Merck Index, 12th Edition (1996), Therapeutic Category and Biological Activity Index, lists therein headed “Analgesic”, “Anti-inflammatory” and “Antipyretic”).

Particularly preferred combination therapies comprise use of a composition of the invention with an opioid compound, more particularly where the opioid compound is codeine, meperidine, morphine or a derivative thereof.

A valdecoxib composition of the invention can also be administered in combination with a second selective COX-2 inhibitory drug, for example celecoxib, rofecoxib, etc.

The compound to be administered in combination with valdecoxib can be formulated separately from the valdecoxib or co-formulated with the valdecoxib in a composition of the invention.

Compositions of the invention are generally suitable for administration of valdecoxib in a daily dosage amount from about 1 mg to about 100 mg. Each dose unit of a composition of the invention typically comprises an amount of valdecoxib from about one-tenth of the daily dosage amount to the whole of a daily dosage amount. Preferred daily dosage amounts are about 2 mg to about 60 mg, more preferably about 5 mg to about 40 mg, for example about 5 mg, about 10 mg, about 20 mg or about 40 mg.

### EXAMPLES

The following examples are provided for illustrative purposes only and are not to be construed as limitations.

### Example 1

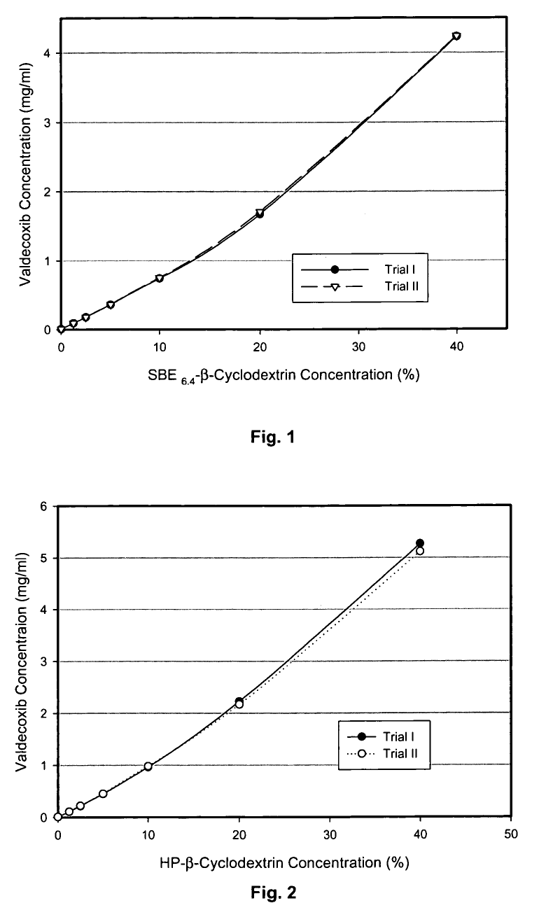

Several buffer solutions were prepared comprising 10 mM disodium phosphate, pH 8.1. In individual tubes, sulfobutylether_{6.4}-β-cyclodextrin was added at concentrations of 1.25, 2.5, 5, 10, 20 or 40% (w/v). Approximately 50 to 100 mg of valdecoxib was added to each tube and the tubes were subjected to vortexing and sonication. Each tube was then placed on a rotating shaker for at least 24 hours at room temperature. Following shaking, all tubes were centrifuged, any valdecoxib sediment which had formed was removed, and supernatant was passed through 0.45 micron disc syringe filters. The filtrate from each tube was then analyzed for valdecoxib concentration using high performance liquid chromatography (HPLC). All tests were performed in duplicate. Data are shown in FIG. 1. Surprisingly, at higher cyclodextrin concentrations, the weight ratio of solubilized valdecoxib to cyclodextrin was greater than at lower cyclodextrin concentrations. For example, at 1.25% cyclodextrin, w/v, the weight ratio of solubilized valdecoxib to cyclodextrin is 7.39 (0.0924 mg valdecoxib/ml composition per 0.0125 mg cyclodextrin/ml composition). On the other hand, at 20% cyclodextrin, w/v, the weight ratio of solubilized valdecoxib to cyclodextrin is 8.45 (1.689 mg valdecoxib/ml composition per 0.20 mg cyclodextrin/ml composition). At 40% sulfobutylether_{6.4}-β-cyclodextrin concentration, valdecoxib solubility is increased approximately 520-fold over the same solution with no cyclodextrin.

### Example 2

Several buffer solutions were prepared comprising 10 mM disodium phosphate, pH 8.1. In individual tubes, hydroxypropyl-β-cyclodextrin was added at concentrations of 1.25, 2.5, 5, 10, 29 or 40% (w/v). The hydroxypropyl-β-cyclodextrin had an average degree of substitution with hydroxypropyl groups of 6.4). Approximately 50 to 100 mg of valdecoxib was added to each tube and the tubes were subjected to vortexing and sonication. Each tube was then placed on a rotating shaker for at least 24 hours at room temperature. Following shaking, all tubes were centrifuged, any valdecoxib sediment which had formed was removed, and supernatent was passed through 0.45 micron disc syringe filters. The filtrate from each tube was then analyzed for valdecoxib concentration using HPLC. All tests were performed in duplicate. Data are shown in FIG. 2. Surprisingly, at higher cyclodextrin concentrations, the ratio of solubilized valdecoxib to cyclodextrin concentration was greater than at lower concentrations. At 40% hydroxypropyl-β-cyclodextrin concentration, valdecoxib solubility is increased approximately 420-fold over the same solution with no cyclodextrin.

### Example 3

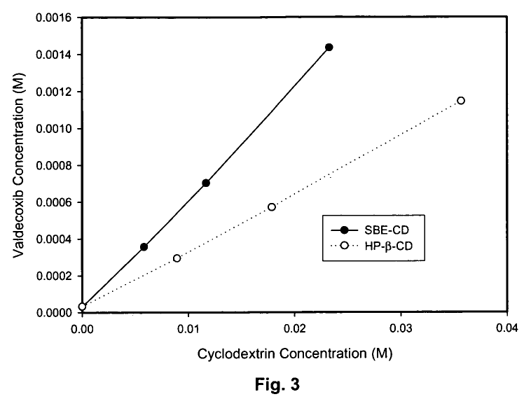

When dilute concentrations of cyclodextrin were used in an experimental procedure substantially as described in Examples 1 and 2, alinear plot of valdecoxib concentration (w/v) versus sulfobutylether_{6.4}-β-cyclodextrin or hydroxypropyl-β-cyclodextrin concentration (w/v) was obtained. Data, shown in FIG. 3, indicate that at low cyclodextrin concentration, valdecoxib and both cyclodextrinb derivatives bind at a 1:1 molar ratio. K_{1:1} binding constants were also determined substantially according to the procedure described in Higuchi, T. & Connors, K. A. Phase Solubility Techniques, Advan. Anal. Chem. Instrum. (1965) 4:117. In general, valdecoxib solubility measured in different cyclodextrin solutions was plotted against cyclodextrin concentration. Slope of the linear portion of the resulting curve was determined. Binding constant for a one-to-one complex between valdecoxib and cyclodextrin, K_{1:1}, was determined using the following equation:

K_{1:1}=slope/Sₒ(1−slope)

where Sₒ is the intrinsic solubility of valdecoxib measured in a control 10 mM disodium phosphate buffer, pH 8.1. Binding constant data are shown in Table 1.

TABLE 1
Cyclodextrin binding constants

| Cyclodextrin Derivative | K_{1:1} at 25° C. (M⁻¹) |
|---|---|
| SBE_{6.4}-CD | 2022 |
| HP_{4.6}-β-CD | 1002 |

As can be seen from the data, SBE_{6.4}-CD binds approximately twice as tightly to valdecoxib as does HP_{4.6}-β-CD.

What is claimed is:

1. A pharmaceutical composition comprising a liquid carrier, valdecoxib; and a cyclodextrin;
wherein the cyclodextrin is present in an amount of not less than 5%, w/v, of the composition, and
wherein at least a substantial portion of the valdecoxib is in solubilized form in the liquid carrier and wherein at a given temperature, the weight ratio of said valdecoxib in solubilized form to said cyclodextrin is in a proportion of at least about 2.5% greater than is achievable in a substantially similar composition but which comprises less than 5%, w/v, of said cyclodextrin at the given temperature.

2. The composition of claim 1 wherein said cyclodexrin amount is not less than about 7.5%, w/v, of the composition.

3. The composition of claim 1 wherein said cyclodexrin amount is not less than about 15%, w/v, of the composition.

4. The composition of claim 1 wherein said proportion is greater than at least about 5.0 %.

5. The composition of claim 1 wherein said proportion is greater than at least about 10.0%.

6. The composition of claim 1 wherein the cyclodextrin is an α-cylcodextrin and/or a β-cyclodextrin.

7. The composition of claim 1 wherein the cyclodextrin is an at least partially etherified β-cyclodextrin.

8. The composition of claim 1 wherein the cyclodextrin is a sulfoalkyl ether cyclodextrin and/or a hydroxyalkyl-β-cyclodextrin.

9. The composition of claim 1 wherein the cyclodextrin is a sulfobutyl ether cyclodextrin and/or hydroxypropyl-β-cyclodextrin.

10. The composition of claim 1 wherein the valdecoxib is present at a concentration of not less than about 1 mg/ml of the composition.

11. The composition of claim 1 wherein the valdecoxib is present at a concentration of not less than about 2 mg/ml of the composition.

12. The composition of claim 1 wherein the valdecoxib is present at a concentration of about 4 mg/ml to about 10 mg/ml.

13. The composition of claim 1 wherein the liquid carrier comprises water suitable for injection.

14. The composition of claim 1 wherein (a) the valdecoxib is present at a concentration of about 4 mg/ml to about 10 mg/m; (b) the cyclodextrin is a sulfobutyl ether cyclodextrin and/or hydroxypropyl-β-cyclodextrin; (c) said cyclodexrin amount is not less than about 7.5%, w/v, of the composition; and (d) said proportion is greater than at least about 5.0%.

15. The method comprising administration of the composition of claim 1 to a subject in need thereof.

16. The method of claim 15 wherein said need is treatment and/or prevention of or from a cyclooxygenase-2 mediated disorder or condition.

17. The method of claim 15 wherein said administration is once or twice daily.
